(19) United States
(12) Patent Application Publication
(10) Pub. No.: US 2008/0183261 A1
(43) Pub. Date: Jul. 31, 2008

(54) FIBROSIS-LIMITING MATERIAL ATTACHMENT

(75) Inventors: Eric Falbe Hammill, Ham Lake, MN (US); Michelle Fangmeier, Blaine, MN (US); James P. Arneberg, Roseville, MN (US)

Correspondence Address: SCHWEGMAN, LUNDBERG & WOESSNER, P.A., P.O. BOX 2938, MINNEAPOLIS, MN, 55402, US

(73) Assignee: CARDIAC PACEMAKERS, INC., ST. PAUL, MN (US)

(21) Appl. No.: 11/669,669

(22) Filed: Jan. 31, 2007

(51) Int. Cl. A61N 1/00

(52) U.S. Cl. 607/119

(57) ABSTRACT

Defibrillator lead designs and methods for improved attachment strength between a fibrosis-limiting material covering, a shocking coil electrode, and an implantable lead body are disclosed herein. In certain examples, a portion of the fibrosis-limiting material extends proximal or distal to a shocking coil end and is disposed between a first and a second lead component. In certain examples, a length of compression tubing is utilized. A chronically implanted lead is often encapsulated by a body's fibrotic reaction, which in turn causes future lead explantation to be exceedingly difficult. To reduce fibrotic entanglement, the fibrosis-limiting material covering surrounds strategic portions of the lead. Improving the attachment between the fibrosis-limiting material covering, the shocking coil electrode, and the lead body will allow for improved performance, durability, and extractability of the lead. This disclosure describes several defibrillator lead designs and methods to create these improved joints.

### TECHNICAL FIELD

This patent document pertains generally to implantable defibrillator leads. More particularly, but not by way of limitation, this patent document pertains to the attachment of fibrosis-limiting material to one or more portions of an implantable defibrillator lead.

### BACKGROUND

Cardiac and other defibrillation systems typically include an implantable medical device (IMD), such as a pulse generator, electrically connected to the heart by at least one implantable defibrillator lead. More specifically, an implantable defibrillator lead provides an electrical pathway between the IMD, connected to a proximal end of the lead, and cardiac tissue, in contact with a distal end of the lead. In such a manner, electrical stimulation (e.g., in the form of one or more shocks or countershocks) emitted by the IMD may travel through the implantable defibrillator lead and stimulate the heart via one or more exposed, helically wound shocking coil electrodes located at or near the lead distal end portion. Once implanted, the exposed shocking coil electrodes often become entangled with fibrosis (i.e., a capsule of inactive tissue which grows into the exposed coils) with the end result being that a chronically implanted lead can be extremely difficult to remove by the application of tensile force to the lead proximal end.

Over time, situations may arise which require the removal and replacement of an implanted defibrillator lead. As one example, an implanted defibrillator lead may need to be replaced when it has failed, or if a new type of cardiac device is being implanted which requires a different type of lead system. As another example, bodily infection or shocking coil electrode dislodgement may require the replacement of an implanted defibrillator lead. In such situations, the implanted defibrillator lead may be removed and replaced with one or more different implantable leads.

To allow for easier removal, some implantable defibrillator leads include a fibrosis-limiting material covering a portion of the one or more otherwise exposed shocking coil electrodes thereon. Unfortunately, current fibrosis-limiting materials are applied to the shocking coil electrodes in ways that lack sufficient attachment strength. As a result, when subjected to shear loads, such as during lead implantation procedures, the fibrosis-limiting material may separate from the associated shocking coil electrode or the shocking coil electrodes themselves may separate from the lead body or deform, thereby leaving uncovered coils that are subject to future fibrotic entanglement.

### SUMMARY

Certain examples of the present subject matter include a lead comprising a lead body, at least one shocking coil electrode, and a fibrosis-limiting material. The lead body extends from a lead proximal end portion to a lead distal end portion and may optionally include an inner insulating layer and an outer insulating layer. At least one shocking coil electrode is disposed at one or both of the lead intermediate portion or the lead distal end portion. The fibrosis-limiting material coaxially surrounds, at least in part, the at least one shocking coil electrode and a portion thereof extends proximal or distal to a shocking coil electrode end. This extending portion of the fibrosis-limiting material can be disposed between a first lead component and a second lead component, such as the inner insulating layer and the outer insulating layer of the lead body, for example.

Certain examples of the present subject matter include a lead comprising a lead body, at least one shocking coil electrode, a fibrosis-limiting material, and a length of compression tubing. The lead body optionally includes an inner insulating layer and an outer insulating layer. The at least one shocking coil electrode is disposed on the lead body and is surrounded, at least in part, by the fibrosis-limiting material. The length of compression tubing extends from a tubing first portion to a tubing second portion. The tubing first portion is disposed over a shocking coil electrode end and the tubing second portion is disposed between a first lead component and a second lead component.

Certain examples of the present subject matter include a method comprising coaxially fitting a fibrosis-limiting material over at least one shocking coil electrode, forming the fibrosis-limiting material onto an outer surface of the at least one shocking coil electrode, coupling one or more portions of the at least one shocking coil electrode to a lead body or component, and disposing an extending portion of the fibrosis-limiting material between a first lead component and a second lead component. The coaxial fitting of the fibrosis-limiting material over the at least one shocking coil electrode includes positioning the extending portion of the fibrosis-limiting material proximal or distal to a shocking coil electrode end.

Advantageously, the present leads and methods decrease the likelihood of moving or shifting between a shocking coil electrode and a fibrosis-limiting material covering thereon or between the shocking coil electrode and adjacent portions of a lead body, such as during the lead implantation process. In this way, there is a reduction or elimination of uncovered, implanted shocking coil electrodes that are subject to future fibrotic entanglement, thereby improving the ease of chronic lead extraction should it become necessary. Additionally, the present leads and methods provide smooth transitions at the lead body-shocking coil electrode interface, which also facilitate lead implantation and extractability. These and other examples, advantages, and features of the present leads and methods will be set forth in part in the detailed description, which follows, and in part will become apparent to those skilled in the art by reference to the following description of the present leads, methods, and drawings or by practice of the same.

### BRIEF DESCRIPTION OF THE DRAWINGS

In the drawings, like numerals describe substantially similar components throughout the several views. Like numerals having different letter suffixes represent different instances of similar components. The drawings illustrate generally, by way of example, but not by way of limitation, various embodiments discussed in the present document.

FIG. 1 illustrates a schematic view of a cardiac defibrillator system, including an IMD and an implantable defibrillator lead, as constructed in accordance with at least one embodiment.

FIG. 2 illustrates a plan view of an implantable defibrillator lead, as constructed in accordance with at least one embodiment.

FIG. 3 illustrates an enlarged cross-sectional view of a portion of an implantable defibrillator lead, such as along line 3-3 of FIG. 2, and an implanted environment, as constructed in accordance with at least one embodiment.

FIGS. 4A-4C illustrate an enlarged cross-sectional view of a portion of an implantable defibrillator lead, such as along line 4-4 of FIG. 2, as constructed in accordance with various embodiments.

FIG. 5A illustrates an exploded view of a portion of an implantable defibrillator lead, such as portion 5A of FIG. 2, as constructed in accordance with at least one embodiment.

FIG. 5B illustrates an enlarged cross-section view of a portion of an implantable defibrillator lead, such as along line 5B-5B of FIG. 5A, as constructed in accordance with at least one embodiment.

FIG. 6 illustrates a schematic view of an implantable defibrillator lead being advanced through an introducer sheath (shown in cross-section), as constructed in accordance with at least one embodiment.

FIG. 7 illustrates a schematic view of an implanted defibrillator lead being extracted from a patient, as constructed in accordance with at least one embodiment.

FIG. 8 illustrates a method of attaching a fibrosis-limiting material to one or more portions of an implantable defibrillator lead, as constructed in accordance with at least one embodiment.

### DETAILED DESCRIPTION

The following detailed description includes references to the accompanying drawings, which form a part of the detailed description. The drawings show, by way of illustration, specific embodiments in which the present leads and methods may be practiced. These embodiments, which are also referred to herein as “examples,” are described in enough detail to enable those skilled in the art to practice the present leads and methods. The embodiments may be combined, other embodiments may be utilized or structural or logical changes may be made without departing from the scope of the present leads and methods. The following detailed description is, therefore, not to be taken in a limiting sense, and the scope of the present leads and methods is defined by the appended claims and their legal equivalents.

In this document, the terms “a” or “an” are used to include one or more than one, and the term “or” is used to refer to a nonexclusive “or” unless otherwise indicated. In addition, it is to be understood that the phraseology or terminology employed herein, and not otherwise defined, is for the purpose of description only and not of limitation.

Fibrosis-limiting material coverings for shocking coil electrodes facilitate the extractability ease of chronically implanted defibrillator leads. Unfortunately, the nature of fibrosis-limiting materials and previous manufacturing methods to attach such materials to the shocking coil electrodes lack in physical strength. As one example, certain fibrosis-limiting materials, such as expanded polytetrafluoroethylene (ePTFE), resist adhesion due to their chemical nature and require extremely high heat to sinter to a shocking coil electrode. This high heat exceeds temperatures that many lead body materials can withstand. For at least this reason, previous shocking coil electrodes that are partially surrounded by a fibrosis-limiting material are typically attached to a lead body solely at their very ends using, for example, a medical adhesive.

It has been found that when implanting such previously manufactured defibrillator leads, high drag forces are created along the lead body (e.g., due to an introducer seal of a hemostatic introducer). As a result, several lead component interfaces, including the fibrosis-limiting material to shocking coil electrode and the shocking coil electrode to lead body, have a tendency to separate or shift relative to one another leaving one or more uncovered coils. Advantageously, the present leads and methods provide improved attachment strength between the fibrosis-limiting material, the shocking coil electrodes, and the lead body, and as a result, reduce or eliminate the presence of uncovered, implanted shocking coil electrodes which are subject to future fibrotic entanglement.

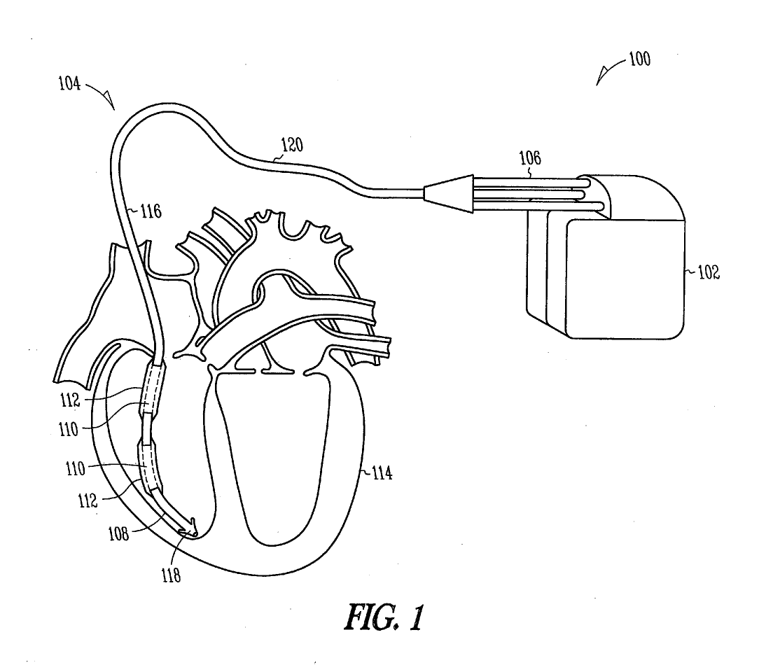

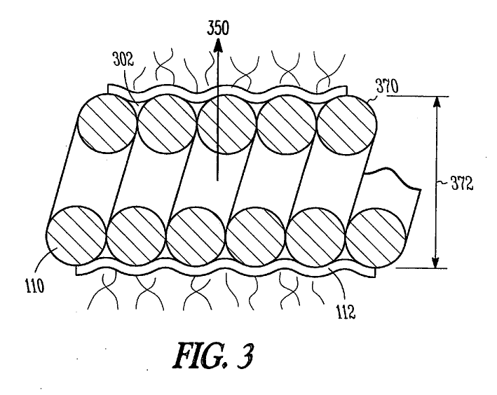

FIG. 1 illustrates a schematic view of a cardiac defibrillator system 100, which is useful for the correction of tachycardia or fibrillation, among other things. The system 100 includes an IMD 102 and at least one implantable defibrillator lead 104. As shown, the implantable defibrillator lead 104 includes a lead body 120 extending from a lead proximal end portion 106, coupled with the IMD 102, to a lead distal end portion 108 implanted within, on, or near a heart 114, with a lead intermediate portion 116 therebetween. The lead intermediate portion 116 or the lead distal end portion 108 includes at least one shocking coil electrode 110 thereon. In this example, the at least one shocking coil electrode 110 is surrounded by a fibrosis-limiting material 112. In various examples, the fibrosis-limiting material 112 comprises a thin, polymeric layer coaxially surrounding and contacting an outer surface 370 (FIG. 3) of the helically wound shocking coil electrode 110.

The implantable defibrillator lead 104 transmits electrical signals between a selected location within, on, or about the heart 114 and the IMD 102, such as to monitor the heart's 114 electrical activity at the selected location or to carry stimulation signals (e.g., one or more shocks or countershocks) to the selected location from the IMD 102. The implantable defibrillator lead 104 may include a fixation assembly, such as one or more tines 118 or a helical coil, to anchor the lead distal end portion 118 at the selected located. The one or more tines 118 may be formed as part of the lead body 120, and thus may include a biocompatible lead body material, such as silicone rubber, polyurethane, polyimide, or a non-porous fluoropolymer.

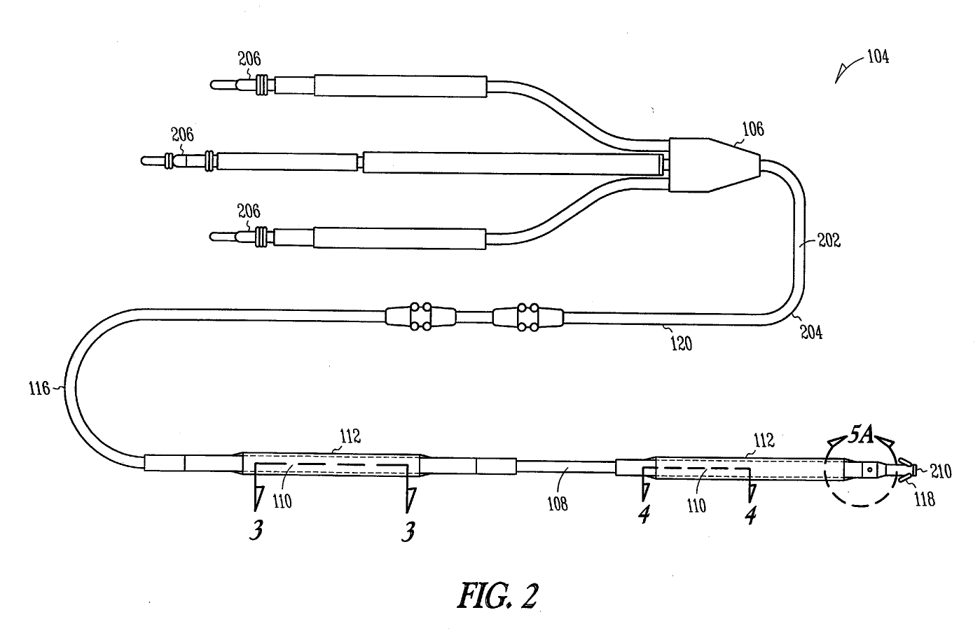

FIG. 2 illustrates a plan view of an implantable defibrillator lead 104. As shown, the implantable defibrillator lead 104 includes a lead body 120 extending from a lead proximal end portion 106 to a lead distal end portion 108 and having a lead intermediate portion 116 therebetween. In various examples, the lead body 120 includes an inner insulator layer 202, such as silicone rubber or other layer of impermeable polymeric electrically insulating material, and an outer insulator layer 204, such as polyurethane which provides high abrasion resistance.

In this example, the lead intermediate portion 116 and the lead distal end portion 108 include a first and a second shocking coil electrode 110. The first and second shocking coil electrodes 110 comprise an uninsulated, helically wound shocking coil formed of a non-corrosive, bio-compatible metal, such as platinum, titanium, or alloys (e.g., platinum/iridium). The shocking coil electrodes 110 are covered by a pliable fibrosis-limiting material 112 (e.g., polytetrafluoroethylene (PTFE) or expanded PTFE (ePTFE)) in direct contact with an outer surface 370 (FIG. 3) of the shocking coil electrode 110. The implantable defibrillator lead 104 of this example further comprises a distal tip electrode 210. The distal tip electrode 210 may be porous and include a metallic mesh. One or more conductors in the lead body 120 electrically and mechanically couple the electrodes 110, 210 to the lead proximal end portion 106. The conductors may be of any structure or combination of structures, such as coaxial or coradial coils separated by an insulating tube, or side-by-side cables or coils separated by a polymer, such as fluoropolymer, silicone, polyimide, or polyurethane.

As shown, but as may vary, the lead proximal end portion 106 includes three terminal leg connections 206 each of which is sized and shaped to couple to respective connector cavities incorporated into a header of the IMD 102 (FIG. 1). It is through the coupling between the lead proximal end portion 206 and the connector cavities that the electrodes 110, 210 are electrically coupled to electronic circuitry within the IMD 102. While FIG. 2 illustrates an implantable defibrillator lead 104 having three terminal connections 206 and three electrodes 110, 210, the present leads may vary, such as by including more or less than three terminal connections 206 and electrodes 110, 210.

FIG. 3 illustrates an enlarged cross-sectional view, such as along line 3-3 of FIG. 2, of a shocking coil electrode 110 surrounded by a thin, fibrosis-limiting material 112. As shown in this example, the fibrosis-limiting material 112 may be drawn into the coil gaps 302, such as via a heat sintering process, thereby eliminating or reducing the air volume present in the gaps. This tight conformation between the fibrosis-limiting material 112 and the shocking coil electrode 110 results in good electrical energy transmission 350 from the coil 110 to surrounding cardiac tissue. The use of the fibrosis-limiting material 112 as the tissue contacting portion of the shocking coil electrode 110 prevents fibrotic tissue ingrowth (as shown), which is often seen as a disadvantage of leads relying on direct contact between an exposed portion of a coil electrode and living tissue.

Options for the fibrosis-limiting material 112 are numerous. For instance, the fibrosis-limiting material 112 may include PTFE, ePTFE, or other non-biodegradable and biocompatible materials, such as expanded ultra-high molecular weight polyethylene (eUHMWPE); may either be porous or non-porous; or may be inherently conductive or rely on porosity in conjunction with bodily fluids to be conductive. In various porous examples, the pore size is adequately small to allow penetration of conductive bodily fluids while substantially precluding tissue ingrowth, thus allowing a less traumatic removal of the defibrillator lead 104 after implantation should extraction become necessary. In various other examples, electrical conductivity through the fibrosis-limiting material 112 is not based on porosity, but rather is inherent in the material 112 as described in commonly-assigned Krishnan, U.S. Pat. No. 7,013,182 titled “CONDUCTIVE POLYMER SHEATH ON DEFIBRILLATOR SHOCKING COIL,” which is hereby incorporated by reference in its entirety.

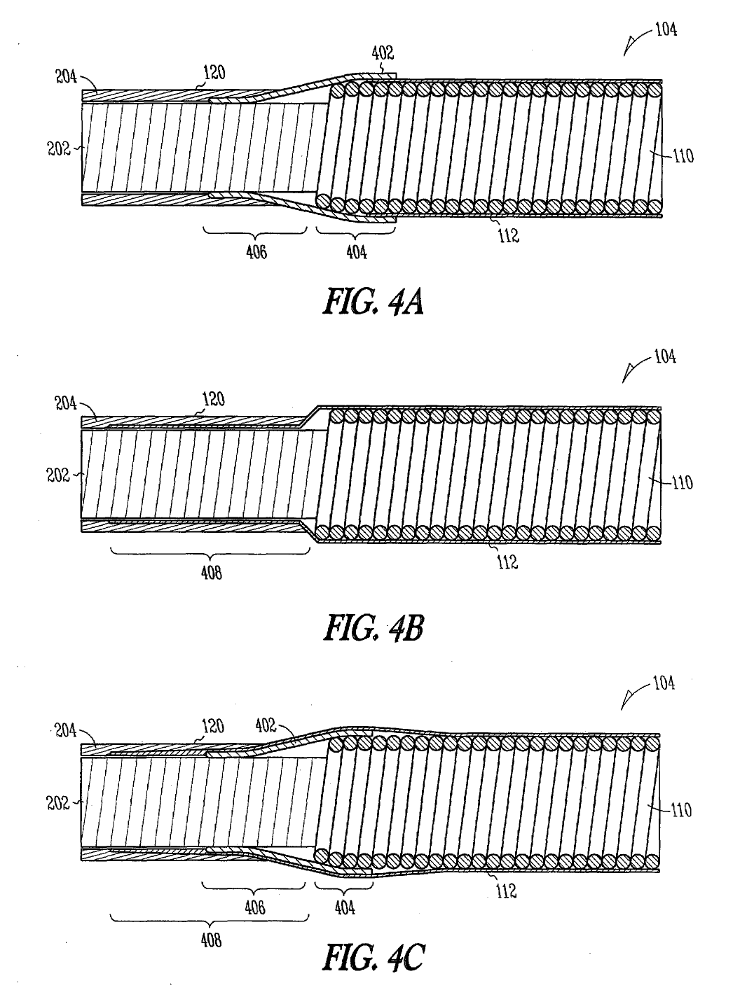

Turning now to FIGS. 4A-4C, various techniques for robustly attaching the fibrosis-limiting material 112 to one or more portions of an implantable defibrillator lead 104 are disclosed. These FIGS. illustrate an enlarged cross-sectional view of an implantable defibrillator lead 104, such as along line 4-4 of FIG. 2, including a lead body 120, a shocking coil electrode 110, and a fibrosis-limiting material 112. The lead body 120 in these examples includes an inner insulator layer 202 and an outer insulator layer 204, the latter of which abuts an end of the helically wound shocking coil electrode 110. As shown, the fibrosis-limiting material 112 coaxially covers portions of the shocking coil electrode 110 in a tightly conforming manner.

In FIG. 4A, a tightly-wound shocking coil electrode 110 is utilized in conjunction with a length of compression tubing 402. The tightly-wound nature of the shocking coil electrode 110 may prevent or reduce axial movement of coil fibers which may occur with a loosely-wound shocking coil (i.e., a coil with spacing between the coil filars); however, the attachment technique of FIG. 4A may also be used with a loosely-wound coil. In this example, a tubing first portion 404 is placed over an end of shocking coil electrode 110, and in some examples, the fibrosis-limiting material 112, while a tubing second portion 406 is disposed between the first 202 and second 204 insulating layers of the lead body 120. In certain examples, the compression tubing 402 comprises silicone or other tubing having an inner diameter smaller than an outer diameter 372 (FIG. 3) of the shocking coil electrode 110 prior to being disposed therearound. In such examples, the tubing 402 is temporarily expanded and placed over the coil 110 and the fibrosis-limiting material 112 end regions, thereby mechanically holding the same in place. In this way, plastic deformation of the shocking coil electrode 110 and relative movement of the fibrosis-limiting material 112 relative to the coil 110 is reduced or eliminated, such as during lead implantation procedures.

FIG. 4B illustrates an attachment technique including a tightly-wound shocking coil electrode 110 in conjunction with a fibrosis-limiting material 112 having one or more ends which extend proximal or distal to an end of the coil 110. While a tightly-wound shocking coil electrode 110 is illustrated, the attachment technique of FIG. 4B may also be used with a loosely-wound coil. The over-extension 408 of the fibrosis-limiting material 112, in this example, is disposed between the inner 202 and outer 204 insulating layers of the lead body 120, thereby holding the ends of the shocking coil electrode 110 and the fibrosis-limiting material 112 in place and not exposing portions of the shocking coil to future fibrotic entanglement. Additionally, stretching of the shocking coil electrode 110 is less likely to occur during lead implantation process using this technique, as an introducer sheath 600 (FIG. 6), specifically an introducer seal 604 (FIG. 6), is less likely to catch and pull a portion of the coil 110.

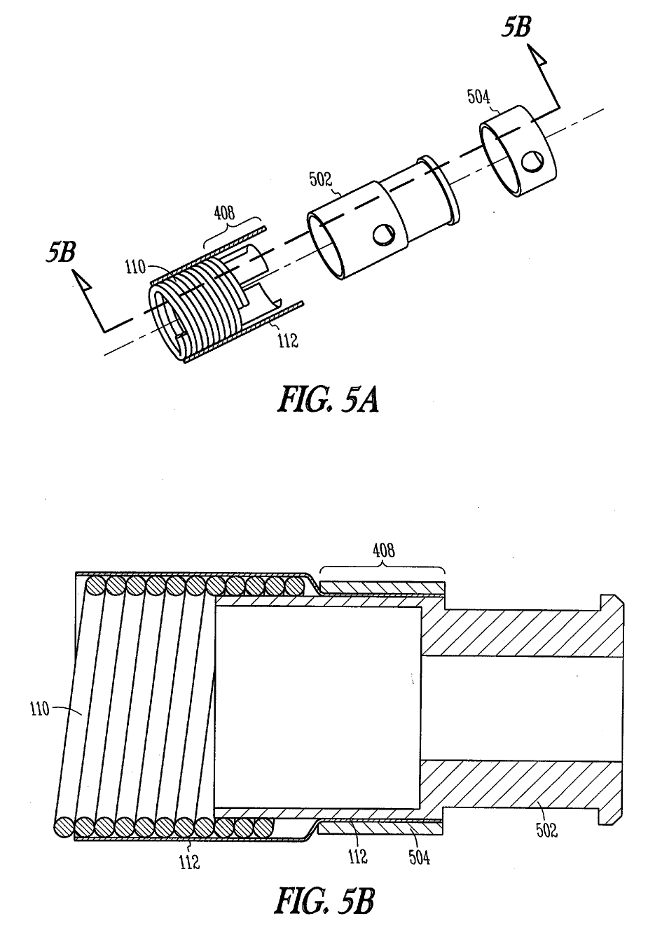

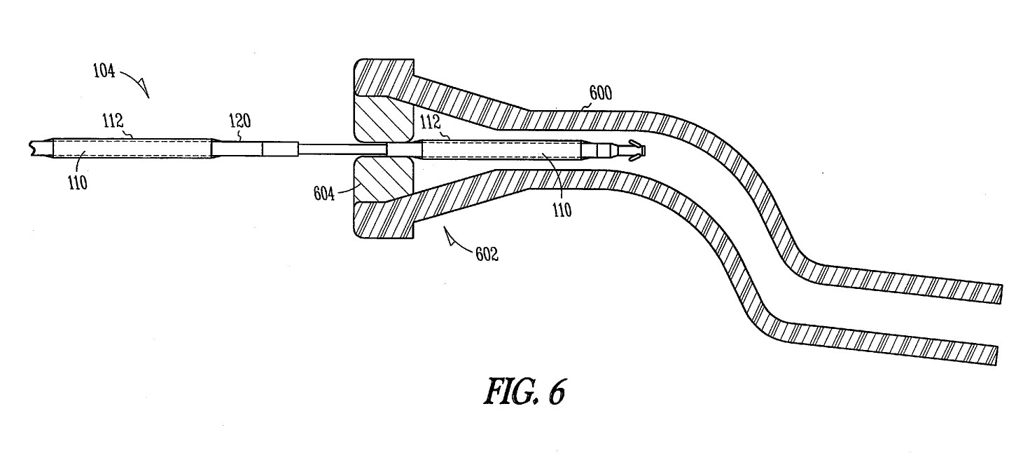

As illustrated in FIGS. 5A and 5B, the over-extension 408 (shown in cross-section) of the fibrosis-limiting material 112 past the shocking coil electrode 110 may alternatively be disposed between two or more crimp or weld rigid (or semi-rigid) structures, such as an annular partial band or ring member 504 and an annular core member 502. In various examples, the annular partial band or ring member 504 and the annular core member 502 are used to secure an over-extension 408 of the fibrosis-limiting material 112 at a distal end of the defibrillator lead 104 (FIG. 2).

In FIG. 4C, a combination of the embodiments shown in FIGS. 4A and 4B is utilized as an attachment technique between the shocking coil electrode 110, the fibrosis-limiting material 112, and the lead body 120. More specifically, the attachment technique of this example includes a tightly-wound shocking coil electrode 110 in conjunction with a length of compression tubing 402 and an over-extending fibrosis-limiting material 112. While a tightly-wound shocking coil electrode 110 is illustrated, the attachment technique of FIG. 4C may also be used with a loosely-wound coil. As shown, a first portion 404 of the compression tubing 402 is placed over an end of shocking coil electrode 110; while a second portion 406 of the compression tubing 402 is disposed between the first 202 and second 204 insulating layers of the lead body 120. The over-extension 408 of the fibrosis-limiting material 112 is disposed between the inner insulator 202 of the lead body 120 and the second portion 406 of the compression tubing, thereby holding the ends of the shocking coil electrode 110 and fibrosis-limiting material 112 in place and not exposing portions of the shocking coil electrode to future fibrotic entanglement. Although not shown, the disposition of the compression tubing 402 and the over-extension 408 of the fibrosis-limiting material 112 may be interchanged.

Advantageously, the techniques shown in FIGS. 4A-4C provide robust mechanical attachment between the fibrosis-limiting material 112, the shocking coil electrode 110, and the lead body 120, yet do not compromise the flexibility of the defibrillator lead 104. In this way, the present attachment techniques allow for long flex life to accommodate interaction with a beating heart 114 (FIG. 1). Prototypes have shown that the manufacturing of these attachment techniques may be accomplished and that the strength of the shocking coil electrode 110 and the fibrosis-limiting material 120 to lead body 120 attachment is improved relative to previously-used attachment schemes.

Implantable defibrillator leads 104 are often placed in contact with cardiac tissue by passage through a venous access, such as the subclavian vein, the cephalic vein, or one of its tributaries. In such a manner, an implantable defibrillator lead 104 may advantageously be placed in contact with the heart 114 (FIG. 1) without requiring major thoracic surgery. Instead, an implantable defibrillator lead 104 may be introduced into a vein and maneuvered therefrom into contact with the heart 114 or tissue thereof. A multi-step procedure is often required to introduce implantable defibrillator leads 104 within the venous system. Generally, this procedure consists of inserting a hollow needle into a blood vessel, such as the subclavian vein. A guide wire is then passed through the needle into the interior portion of the vessel and the needle is withdrawn. As illustrated in FIG. 6, an introducer sheath 600 with a dilator assembly 602 may be inserted over the guide wire into the vessel for lead 104 introduction. The sheath 600 is advanced to a suitable position within the vessel, such that a distal end thereof is well within the vessel, while a proximal end thereof is outside the patient.

When a physician implants a defibrillator lead 104, such as through the introducer sheath 600 and specifically an introducer seal 604, high drag forces may be created along the lead body 120. As a result of these high drag forces, previous lead component interfaces including the fibrosis-limiting material 112 to shocking coil electrode 110 and the shocking coil electrode 110 to the lead body 120 tended to separate or shift relative to one another leaving uncovered coil portions subjected to future fibrotic entanglement (e.g., the shocking coil electrode 110 became stretched, which in turn pulled the fibrosis-limiting material 112 away from the coil 110 and exposed a portion of the coil to fibrotic growth). Using the present attachment techniques, it has been found that such separating or shifting between the fibrosis-limiting material 112, the shocking coil electrode 110, and the lead body 120 is reduced or eliminated, thereby preventing fibrotic entanglement and facilitating lead extraction should it become necessary.

FIG. 7 illustrates a lead extraction device 700. In this example, the lead extraction device 700 includes a weight 702 coupled over a pulley 704 to a proximal end 106 of an implanted defibrillator lead 104 to be removed from a patient 706. By minimizing or preventing the fibrotic entanglement with the shocking coil electrode 110 (FIG. 3), the implanted defibrillator lead 104 may be removed from the patient 706 with relatively small amounts of tensile force (e.g., applied via the weight 702) and reduced time. The lead removal process is therefore relatively atraumatic and is considered to be easily extracted from the patient 706 within which it has been implanted.

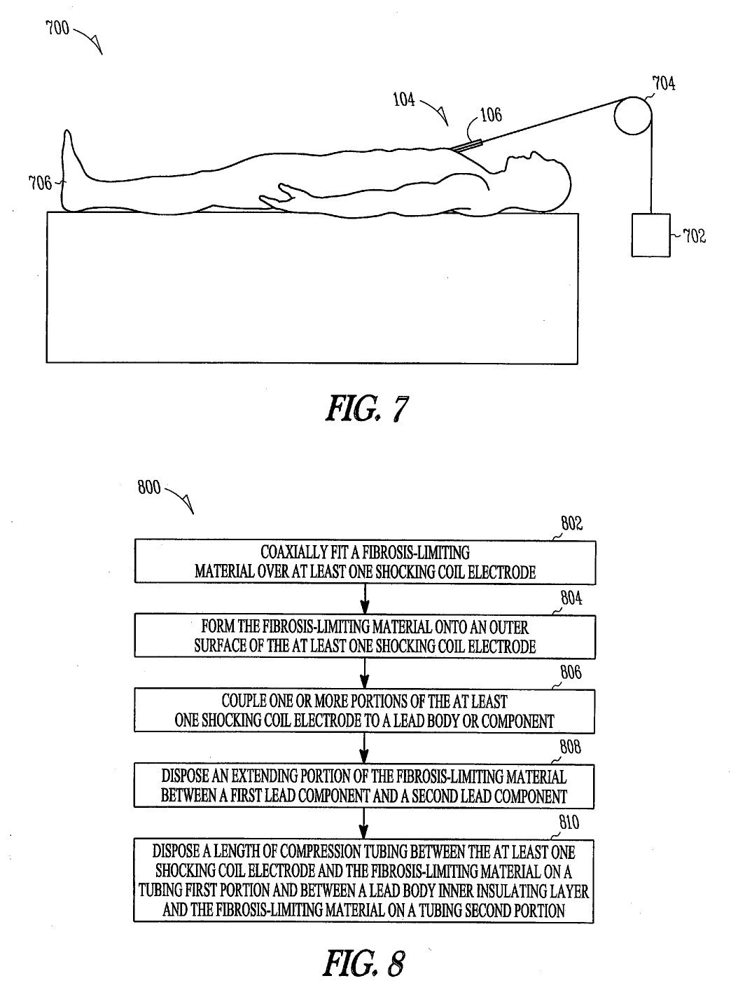

FIG. 8 illustrates a method 800 of manufacturing an implantable defibrillator lead including secure, robust attachment between a fibrosis-limiting material, a shocking coil electrode, and a lead body. At 802, a fibrosis-limiting material is coaxially fit over at least one shocking coil electrode. In various examples, this coaxially fitting includes positioning a portion of the fibrosis-limiting material proximal or distal to a shocking coil electrode end. At 804, the fibrosis-limiting material is formed onto an outer surface of the at least one shocking coil electrode, such as through the use of heat. At 806, one or more portions, such as end portions, of the at least one shocking coil electrode are coupled to a lead body or component. Optionally, the coupling between the shocking coil electrode and the lead body includes the use of an adhesive.

At 808, the proximal or distal portion of the fibrosis-limiting material is disposed between a first lead component and a second lead component. In one example, this disposition between the first and the second lead component includes disposing the fibrosis-limiting material between a lead body inner insulating layer and a lead body outer insulating layer. In another example, the disposition between the first and the second lead component includes disposing the fibrosis-limiting material between a rigid or semi-rigid core member and a rigid partial band or ring member sized and shaped to couple around the rigid core member. Optionally, at 810, a length of compression tubing is disposed between the at least one shocking coil electrode and the fibrosis-limiting material on a tubing first portion and between a lead body inner insulating layer and the fibrosis-limiting material on a tubing second portion.

Leads and methods for improved attachment strength between a fibrosis-limiting material, a shocking coil electrode, and a lead body are discussed. Advantageously, the present leads and methods decrease the likelihood of moving or shifting between such components and, in this way, reduces or eliminates the presence of uncovered, implanted shocking coil electrodes subjected to future fibrotic entanglement. Additionally, the present leads and methods provide smooth transitions at the lead body-shocking coil electrode interface, which facilitate lead implantation and extractability.

It is to be understood that the above description is intended to be illustrative, and not restrictive. For instance, any of the aforementioned examples may be used individually or with any of the other examples. In addition, the aforementioned examples may or may not include the use of adhesives (e.g., medical adhesives) for selected component attachment. Many other embodiments may be apparent to those of skill in the art upon reviewing the above description. The scope of the present leads and methods should, therefore, be determined with reference to the appended claims, along with the full scope of legal equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, assembly, article, or process that includes elements in addition to those listed after such a term in a claim are still deemed to fall within the scope of such claim.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, various features may be grouped together to streamline the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus the following claims are hereby incorporated into the Detailed Description, with each claim standing on its own as a separate embodiment.

What is claimed is:

1. A lead comprising:
a lead body extending from a lead proximal end portion to a lead distal end portion and having a lead intermediate portion therebetween, the lead body having an inner insulating layer and an outer insulating layer;
at least one shocking coil electrode disposed at one or both of the lead intermediate portion or the lead distal end portion; and
a fibrosis-limiting material coaxially surrounding, at least in part, the at least one shocking coil electrode, a portion of the fibrosis-limiting material extending proximal or distal to a shocking coil electrode end and disposed between a first lead component and a second lead component.

2. The lead of claim 1, wherein the first lead component includes the inner insulating layer of the lead body and the second lead component includes the outer insulating layer of the lead body.

3. The lead of claim 1, wherein the first lead component includes a rigid core member having a core outer diameter and the second lead component includes a rigid or semi-rigid ring member having a ring inner diameter, the ring inner diameter being substantially equal to the core outer diameter.

4. The lead of claim 1, wherein an inner surface of the fibrosis-limiting material conforms to an outer surface of the at least one shocking coil electrode.

5. The lead of claim 1, wherein a portion of the fibrosis-limiting material is disposed within one or more gaps of the at least one shocking coil electrode.

6. The lead of claim 1, wherein the fibrosis-limiting material comprises one or more of polytetrafluoroethylene, expanded polytetrafluoroethylene, or expanded ultra-high molecular weight polyethylene.

7. The lead of claim 1, wherein the fibrosis-limiting material includes one or more pores.

8. The lead of claim 1, wherein the fibrosis-limiting material is electrically conductive.

9. The lead of claim 1, wherein the lead body is substantially isodiametric.

10. The lead of claim 1, further comprising a fixation assembly disposed at or near the lead distal end portion.

11. A lead comprising:
a lead body including an inner insulating layer and an outer insulating layer;
at least one shocking coil electrode disposed on the lead body;
a fibrosis-limiting material surrounding and contacting a portion of the at least one shocking coil electrode; and
a length of compression tubing extending from a tubing first portion to a tubing second portion, the tubing first portion disposed over a shocking coil electrode end and the tubing second portion disposed between a first lead component and a second lead component.

12. The lead of claim 11, wherein the first lead component includes the inner insulating layer of the lead body and the second lead component includes the outer insulating layer of the lead body.

13. The lead of claim 11, wherein the first lead component includes a rigid core member and the second lead component includes a rigid or semi-rigid ring member sized and shaped to couple around the rigid core member.

14. The lead of claim 11, wherein the tubing first portion comprises an inner diameter smaller than an outer surface of the at least one shocking coil electrode prior to being disposed therearound.

15. The lead of claim 11, wherein a portion of the fibrosis-limiting material extends proximal or distal to the shocking coil electrode end and is disposed between the length of compression tubing and the outer insulating layer of the lead body.

16. The lead of claim 11, further comprising one or more tines located near a lead distal end portion.

17. The lead of claim 11, further comprising a distal tip electrode.

18. A method comprising:
coaxially fitting a fibrosis-limiting material over at least one shocking coil electrode, including positioning a portion of the fibrosis-limiting material proximal or distal to a shocking coil electrode end;
forming the fibrosis-limiting material onto an outer surface of the at least one shocking coil electrode, including applying heat to the fibrosis-limiting material;
coupling one or more portions of the at least one shocking coil electrode to a lead body or component; and
disposing the proximal or distal portion of the fibrosis-limiting material between a first lead component and a second lead component.

19. The method of claim 18, wherein disposing the proximal or distal portion of the fibrosis-limiting material includes disposing the fibrosis-limiting material portion between a lead body inner insulating layer and a lead body outer insulating layer.

20. The method of claim 18, wherein disposing the proximal or distal portion of the fibrosis-limiting material includes disposing the fibrosis-limiting material portion between a rigid core member and a rigid or semi-rigid ring member.

21. The method of claim 18, wherein coupling the one or more portions of the at least one shocking coil electrode to the lead body includes using an adhesive.

22. The method of claim 18, further comprising disposing a length of compression tubing between the at least one shocking coil electrode and the fibrosis-limiting material on a tubing first portion and between a lead body inner insulating layer and the fibrosis-limiting material on a tubing second portion.
